(19) United States
(12) Patent Application Publication
(10) Pub. No.: US 2003/0130625 A1
(43) Pub. Date: Jul. 10, 2003

(54) Infusion system

(76) Inventors: James D. Jacobson, Lindenhurst, IL (US); Tuan Bui, Green Oaks, IL (US); Qui Chau, Skokie, IL (US); Francis C. Kowalik, Deerfield, IL (US)

Correspondence Address: Francis C. Kowalik, Esq., Corporate Counsel, Law Department, BAXTER INTERNATIONAL INC., One Baxter Parkway, DF3-2E, Deerfield, IL, 60015, US

(21) Appl. No.: 10/040,887

(22) Filed: Jan. 7, 2002

(51) Int. Cl. A61M 5/00

(52) U.S. Cl. 604/253; 604/260

(57) ABSTRACT

A medical fluid control system (10) is in the form of a medical line-set adapted to deliver a medication from a container (20) to a patient. The line-set has a tubing (12) having a MEMS element (14) attached thereto. In a preferred embodiment, the MEMS element (14) is a MEMS pump (14). The tubing (12) has a first end (16) that is connected to the container (20) and a second end (18) that is connected to the patient. The MEMS pump (14) can be controlled by an external controller (38) that is operably connected to the MEMS pump (14) to deliver medication from the container (20), through the tubing (12), and to the patient.

### TECHNICAL FIELD

The present invention generally relates to a medical fluid flow control system such as an infusion system, and more particularly to a method and apparatus for control of such systems using a micro-electromechanical element.

### BACKGROUND OF THE INVENTION

Generally, medical patients require precise delivery of either continuous medication or medication at set periodic intervals. Medical fluid flow control systems that include medical pumps have been developed to provide controlled drug infusion. Using the pump, the drug can be administered at a precise rate that keeps the drug concentration within the therapeutic margin and out of a possible toxic range with certain drugs. These high priced medical pumps provide appropriate drug delivery to the patient at a controllable rate that does not require frequent medical attention.

These pumps are often part of an infusion system that is typically used to deliver medication to a patient. In the case of chronic pain, an infusion system is used when oral or topical medications fail to provide effective pain relief or cause uncomfortable side effects. An infusion system may also be used when delivering medication to a specific site or organ is more effective or causes fewer uncomfortable side effects than delivering the medication systematically to the entire body. The use of an infusion system allows a physician to target sites within the body for more effective delivery of a medication. The infusion system can deliver medication to a patient at a controlled rate as prescribed by a physician.

A medical fluid flow control system can be an infusion system wherein a medication is delivered to a patient, or a draw-type system wherein a fluid is taken from a patient and delivered to a separate container. The system typically includes several different components including tubing, a pump, a reservoir and access port. The system could also have other components such as valves and sensors. The components of the system must remain sterile. Some components such as the tubing, container and access port are typically disposable. Other components may be durable or reusable elements such as the pump, valves and any required electronic controllers or power supplies. These components are typically bigger, expensive pieces of equipment. These components must also be sterilized prior to their next use. This can be expensive and time-consuming. Furthermore, as the pump is often the most costly reusable element of the system, there is increased pressure to use a pump that is less costly and smaller in size, but that can still deliver a medication in a controlled, accurate manner.

Thus, it is desirable to have a medical fluid flow control system that uses as many disposable elements as possible. These components are typically less expensive and do not require repeated sterilization as they can simply be discarded. Such a system also reduces maintenance concerns.

The present invention is provided to solve these and other problems.

### SUMMARY OF THE INVENTION

The present invention is generally directed to a medical fluid control system.

According to a first aspect of the invention, the system preferably includes a length of tube and a micro-electromechanical system (MEMS) element operably connected to the tube. In one preferred embodiment, the element is a MEMS pump. The system can be disposable and implemented with a reusable controller and power source. Other additional elements that may be included in the system are flow valves, flow sensors, and pressure sensors.

According to another aspect of the present invention, a wireless controller is provided to control the MEMS element. The controller may control the element from a remote location.

Other advantages and features of the present invention will be apparent from the following description of the embodiments illustrated in the accompanying drawings.

### BRIEF DESCRIPTION OF DRAWINGS

FIG. 1 is a schematic diagram of an embodiment of a medical fluid flow control system where a micro-electromechanical system (MEMS) element is connected to a line-set;

FIG. 2 is a schematic diagram of another embodiment of the medical fluid flow control system where a MEMS element and other components including a controller are connected to a line-set in another configuration;

FIG. 3 is a schematic diagram of another embodiment of the medical fluid flow control system where a power source is connected to the line-set and is operably connected to a MEMS pump;

FIG. 4 is a schematic diagram of another embodiment of the medical fluid flow control system where MEMS element communication with the controller is wireless; and

FIG. 5 is a schematic diagram of another embodiment of a medical fluid flow control system where the system can be implanted in a body.

### DETAILED DESCRIPTION

While this invention is susceptible of embodiments in many different forms, there is shown in the drawings and will herein be described, in detail, preferred embodiments of the invention. The present disclosure is to be considered as an exemplification of the principles of the invention and is not intended to limit the broad aspect of the invention to the embodiments illustrated.

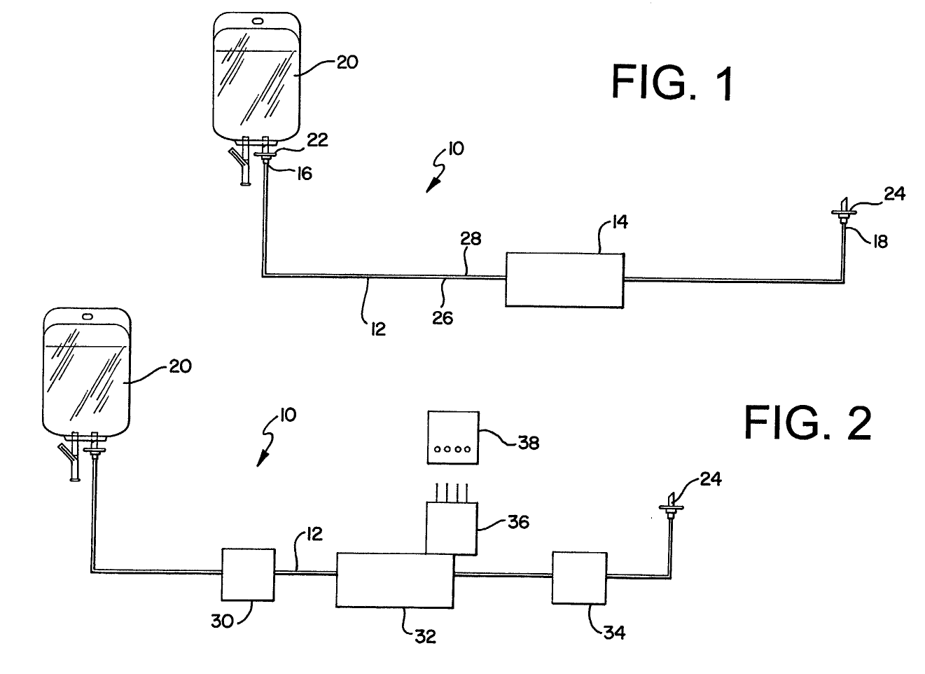

Referring to the drawings, FIG. 1 discloses a medical fluid flow control system of the present invention, generally referred to with the reference numeral 10. The medical fluid flow control system 10 can be configured as an infusion system wherein, for example, a liquid medication is delivered by the system 10 to a patient. It is understood, however, that the system 10 can also be configured as a draw system wherein fluid is taken from a patient and delivered to a container. The medical fluid flow control system 10, in one preferred embodiment, may be in the form of a line-set. The line-set is preferably designed for single use only, disposable after use by patients. The system 10 generally includes a section of tubing 12 and a micro-electromechanical system (MEMS) element 14.

The tubing 12 has a first end 16 and a second end 18. The first end 16 of the tubing 12 is adapted to be connected to a fluid source (a first component) such as an IV bag 20 or other type of reservoir or container. The first end 16 may have a separate connector 22 to connect to the bag 20. The second end 18 of the tubing 12 is adapted to be in communication with, for example, a patient. To that end, the second end 18 may be equipped with an access device 24. The access device 24 can be in the form of a connector for attachment to, for example, a cannula, catheter, syringe, IV line, or any of several other known medical instruments or devices (a second component). The tubing 12 has a generally cylindrical wall 26 defining an interior passageway therethrough 28.

The tubing 12 can be of any suitable medical grade tubing used for procedures requiring a transfer of fluid from at least one source site to at least one recipient site. Exemplary tubing is described in U.S. patent application Ser. No. 08/642,278, entitled “Method of Using Medical Tubings in Fluid Administration Sets,” and U.S. Pat. No. 6,129,876, entitled “Heat Setting of Medical Tubing,” each filed on May 3, 1996, and assigned to the Assignee of this application. Each of these documents is hereby incorporated by reference.

As further shown in FIG. 1, the micro-electromechanical system (MEMS) element 14 is connected to the tube 12. MEMS is a technology used to create tiny devices which can be less than a millimeter in size. MEMS elements are typically fabricated from glass wafers or silicon, but the technology has grown far beyond its origins in the semiconductor industry. Each device is an integrated micro-system on a chip that can incorporate moving mechanical parts in addition to optical, fluidic, electrical, chemical and biomedical elements. The resulting MEMS elements are responsive to many types of input, including pressure, vibration, chemical, light, and acceleration. These devices are smaller than conventional machines used for sensing, communication and actuation. As a result, it is possible to use them in places where mechanical devices could not be traditionally used. MEMS devices also work at a higher rate and consume less power than conventional devices.

The MEMS element 14 can be a number of different components including various types of pumps, a flow valve, a flow sensor, tubing, a pressure sensor or combinations of elements. Because of the actual size of the MEMS element 14, it is understood that the MEMS element 14 is shown schematically in the figures. The MEMS element 14 may be powered by a battery, power supply, or other source of power if necessary. The embodiment shown in FIG. 1 has the source of power and controller as part of the MEMS element 14. As described below, the power source may be separate from the MEMS element 14. The position of the fluid source 20 indicates that gravity may affect the flow within the line-set.

In one preferred embodiment of the system 10, the MEMS element 14 is a MEMS pump 14. As discussed, the MEMS pump 14 in FIG. 1 has an integral power supply. The MEMS pump 14 is capable of pumping fluid contained in the IV bag 20 through the tube 12, out through the access device 24, and into a patient. Once the medication delivery is complete, the system 10 (the tube 12 and MEMS pump 14) can be discarded. It is understood that the IV bag 20 and access device 24 could be considered as parts of the system 10 and can be disposable.

The medical fluid flow control system 10 is capable of many configurations. Additional elements, including MEMS elements 14, can be added to the system 10. FIG. 2 shows the system 10 with additional elements. Similar elements will be referred to with like reference numerals.

In this form, a MEMS pump 32 is connected to the tubing 12. The MEMS pump 32 has a MEMS local electronics element 36 attached thereto. The MEMS electronics element 36 connects with an external, durable MEMS controller 38. As described in greater detail below, a MEMS flow sensor 30 and a MEMS valve element 34 are also connected to the tubing 12. In a preferred form of the MEMS pump 32, the MEMS electronics element 36 is embedded therein and can preferably store MEMS parametric operational information. The MEMS controller 38, with its electronics and power source, are physically connected to the MEMS electronics element 36. Thus, alternatively, the parametric operational information may be loaded from the detachable MEMS controller 38. In another embodiment, the power source may also originate from the MEMS controller 38. It is understood that the power source could be a MEMS element power source or a power source in other forms known in the art. The MEMS controller 38 may be functionally coupled to the MEMS electronics 36 by a variety of methods including the plug type connection depicted. The system may contain one or multiple electrical connection sites 36 for interface to the durable MEMS controller 38. The MEMS electronics 36 may then be used to locally govern the mechanics of the MEMS pump 32.

The flow sensor 30 can be added to the system 10 to enable more accurate fluid delivery. The flow sensor 30 could also take the form of a pressure sensor if desired. The valve element 34 could alone be added to the typical system to allow metering from a pressurized or otherwise forced system. The flow sensor 30 and valve 34 can assist in controlling the rate of flow and the direction of flow in micro-fluidic circuits and devices in conjunction with the MEMS pump 32. If desired, the system may also include a slide clamp or other more traditional auxiliary features. A slide clamp may be particularly useful to manually occlude flow in the case of an alarm indicating pump malfunction in a case where the MEMS componentry is normally open. These MEMS elements could be fabricated as one monolithic unit to be added to the system 10 or as separate elements.

The delivery process may implement a normally closed valve 34 or pump 32 designed to open and allow fluid flow only upon sufficient power and appropriate communication transfer to the local electronics element 36 from the controller 38, thereby providing a no-flow condition without the use of cumbersome mechanical devices. This normally closed feature may be integrated directly within other MEMS componentry such as the pump 32 or as a separate MEMS element.

Preferably, the pump element 32 generates the fluid flow through a tube 12 based on information stored locally within the MEMS electronics 36. This information is preferably downloaded from the detachable MEMS controller 38. The direction of fluid flow is preferably from the fluid source 20 into the first tube end 16, directed by the pump 32, through the second tube end 18 to the access device 24 as in medical infusion. In medical infusion configurations, the access device 24 is typically a catheter or needle. The source of fluid in medical infusion devices is generally the IV bag 20 or some type of container. The pump element 32 is instructed by the local MEMS electronics 36 to deliver a controlled amount of medication through the tube 12 to a patient. In the system configuration shown in FIG. 2, the sole reusable element is the controller 38 while the remaining elements can be disposable. The controller 38 can control the pump element 32 in a variety of different ways. It can supply intermittent power or power such that the pump element 32 will run in a “slow mode” or a “fast mode.” The controller 38 can supply the power and instructions to the pump element 32 as desired.

Fluid could potentially be directed to flow in the opposite direction. In this embodiment, fluid is drawn by the access device 24, into the second end 18 of the tube 12, due to the action of the pump element 32, with its valves 34 and sensors 30, through the first end 16 of the tube 12, and into the reservoir 20. The medical fluid flow control system 10, in this draw configuration, can be preferably regulated by the use of the pump controller 38 that is electrically connectable to the pump electronics element 36.

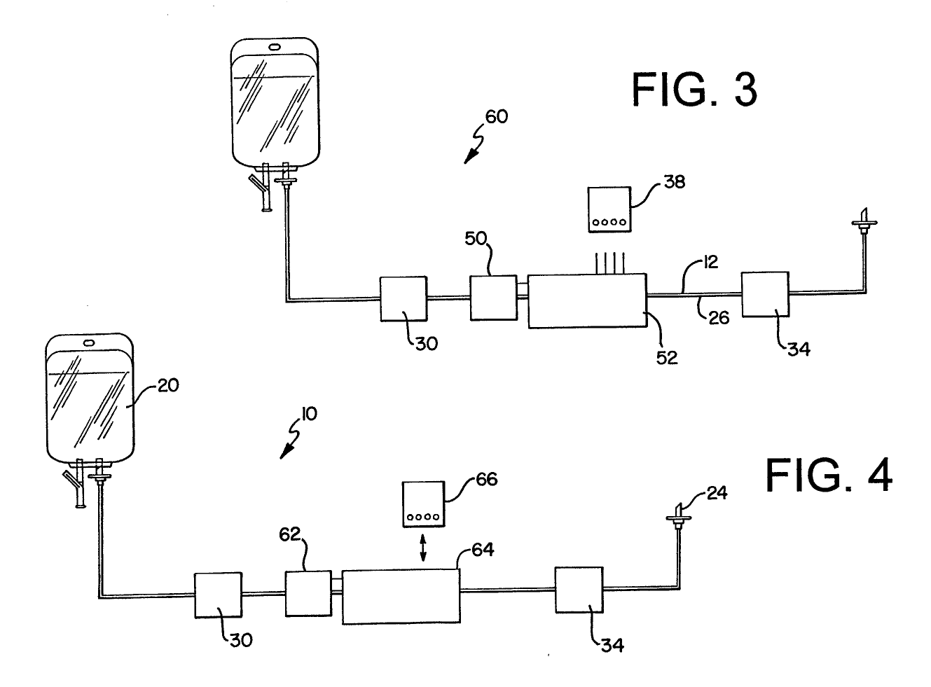

Referring now to FIG. 3, there a diagram of yet another embodiment of the present invention. A power source 50 such as a small battery, fuel cell, or other power supply is added to the system 10 to further decrease the amount of functionality within the durable controller element 38. The power source 50 is preferably connected to the tubing 12 and operably connected to a MEMS pump element 52 similar to the MEMS pump element 32. The power source 50 is designed to last for the life of the MEMS portion of the system. In one embodiment utilizing a fuel cell, the fuel cell 50 is provided as an integral component to an outer surface of the tubing 12. By integral it is meant that the fuel cell 50 is permanently attached to the tubing surface 26 by any suitable means. The power source 50 will also have any necessary activating structure to commence the supply of power. The fuel cell 50 may be any of a myriad of fuel cell designs available and suitable for such use with a line-set such as disclosed in commonly-owned U.S. patent application Ser. No. ______, Attorney docket number 99-6624 (1417 G P 446) entitled “Medical Infusion System with Integrated Power Supply and Pump Therefore,” filed concurrently herewith and expressly incorporated by reference herein. While the power supply 50 is shown in FIG. 3 as connected to the MEMS pump 52, it is understood that the power supply 50 could be operably connected to other components as desired.

The use of MEMS or other emerging economical fabrication techniques provides an opportunity to add a MEMS element to a disposable line-set that provides additional functionality such as pumping, valving, and sensing. Some or all of the supporting local electronics could be included in a disposable portion of a line-set as well. For example, it may be preferable to include a memory chip that contains calibration information for a pump 52, pressure sensor and/or flow sensor 30, valve 34, or a combination of disposable elements. Disposability is desirable as it removes the need for costly sterilization of the components of the system between each subsequent application.

The durable controller 38 is designed to stimulate fluid distribution quantities directly to the MEMS element 52. This type of controller 38 can be utilized for multiple applications, thus making it reusable. The controller 38 would need minimal alterations for similar reapplication. For example, the dosage for a new patient must be reconfigured by the MEMS element 52 via the reusable controller 38. Such a line-set may in fact be a complete infusion and extrusion system contained in a very small package.

In a preferred embodiment shown in FIG. 3, the MEMS pump element 52 would contain electrical connectivity to enable interface to the durable controller 38 that would control the pump 52 to maintain a desired flow rate. The MEMS pump element 52 can be disposed of with the rest of the disposable components of line-set. The electronics of the controller 38 and any type of case or user's interface would be maintained as a durable, reusable system.

Turning now to FIG. 4, there is pictured a schematic diagram of still another embodiment of the present invention. In this configuration, the system 10 may utilize wireless communication. A MEMS pump 64 is connected to the tube 12. A power supply 62 is connected to the tube and is operably connected to the pump 64. A wireless controller 66 is provided to control the MEMS pump 64. Wireless communication removes the previous requirement of developing electrical connectivity for the disposable line-set. A wireless linkage will also reduce the complexity of the line-set usage since it will not need to be loaded in as specific a manner as would be the case with hard wired electrical connections. Wireless communication linkage also provides flexibility in terms of usage, for example allowing a disposable, implantable MEMS pump 64 to be controlled by an external system controller 66. It is understood that in a wireless configuration, the MEMS pump 64 will be equipped with appropriate support structure such as to collect energy transmissions and translate power/control to the pump.

In this configuration, the durable, or reusable, wireless controller 66 would communicate via an inductive or capacitive wireless link, with the MEMS pump 64. It is understood that wireless communication could be established with other MEMS components. The MEMS pump 64, or other MEMS components would be disposable but would be provided with the necessary power and electronics to function properly. For example, the disposable elements may require electronics to support the transfer of information from the disposable elements back to the durable controller 66. It is preferable, however, to include as much of the electronics as possible in the durable controller 66 rather than with disposable elements. It may be desirable to maintain sufficient electronics on the disposable side to accept, store, and interpret packets of instruction sets and power so as to reduce required real-time interaction between the durable and disposable portions of the system.

The durable system controller 66 may in turn provide a transfer of information to and from a LAN or other network to fully automate the control and interrogation of the MEMS element 64 into an automated information management system. Optimally, system control and parametric adjustments can be achieved by wireless communication from and to a MEMS system controller 66.

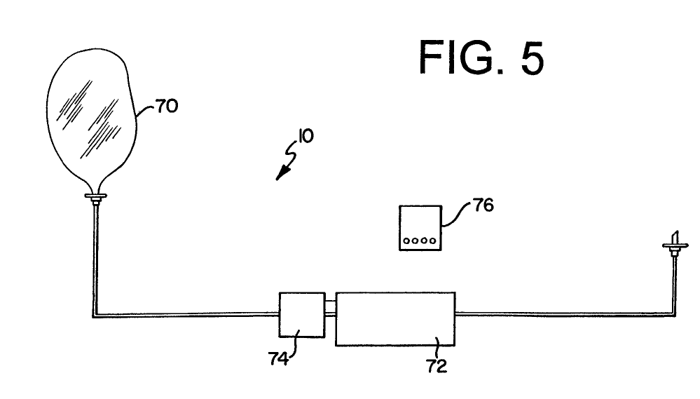

FIG. 5 discloses another embodiment of the medical fluid flow control system 10 of the present invention wherein the system 10 is designed to be implantable within a body. The system 10 utilizes a fluid source or reservoir 70 that is substantially smaller than a conventional IV bag and is disposable. Preferably, a MEMS pump element 72 is connected to the tubing 12. The MEMS pump element 72 has a power supply 74 connected thereto. A wireless controller 76, designed to be remote from the body, communicates wirelessly with the MEMS pump element 72. Thus, all components of the system 10 in FIG. 5 except the controller 76 are designed to be implanted in the body. The durable wireless controller 76 provides the system with the parametric data that the local electronics of the MEMS pump element 72 needs to perform infusion or extrusion.

The fluid reservoir 70 may be refillable and the disposable pieces of the system may include other components such as MEMS valves 34 or sensors 30. Significant advantages over existing methodology include the transfer of mechanical features from a durable system to a disposable portion of the system. This design allows for cheaper construction of the pump controller 76 or durable system 76 and longer-term reliability since the durable system 76 would not include mechanical components. This system also provides the opportunity to develop completely disposable systems or durable/disposable platforms of various fashions.

In another embodiment, the pump 72 itself rather than the reservoir 70 may store and release prescribed amounts of medication into the body. In applications such as an implantable system, there may be no need for an access device 24 in the line-set. A hole or port in the pump 72 may be sufficient to provide a medication exit site from the implanted MEMS system.

The medical fluid flow control system 10 of the present invention may be used when more traditional therapies are considered ineffective or inappropriate. In the case of chronic pain, an infusion and extrusion system is used when oral, intravenous, or topical medications fail to provide effective pain relief or cause uncomfortable side effects. An infusion and draw system can commonly be used when delivering the medication to a specific site or organ is more effective or causes fewer uncomfortable side effects than delivering the medication systemically (to the entire body). The use of a medical fluid flow control system allows a physician to target sites within the body for more effective delivery of a medication. The use of MEMS technology allows more portions of the system 10 to be disposable thus reducing the costs of the system 10. With the use of a MEMS pump having an integral power supply wherein the pump is designed to operate at a single desirable flow rate, a separate durable controller can be eliminated. Thus, an entire infusion system can be designed from disposable components.

While the specific embodiments have been illustrated and described, numerous modifications can be made to the present invention, as described, by those of ordinary skill in the art without significantly departing from the spirit of the invention. The breadth of protection afforded this invention should be considered to be limited only by the scope of the accompanying claims.

We claim:

1. A line-set comprising:
a length of tube; and
a micro-electromechanical system (MEMS) element connected to the tube.

2. The line-set of claim 1 further comprising a controller operably connected to the MEMS element.

3. The line-set of claim 1 wherein the MEMS element is a flow sensor.

4. The line-set of claim 1 wherein the MEMS element is a flow valve.

5. The line-set of claim 1 wherein the MEMS element is a pressure sensor.

6. The line-set of claim 2 wherein the controller is detachable from the MEMS element.

7. The line-set of claim 2 wherein the controller has a means for storing information.

8. The line-set of claim 2 wherein the controller has a means for displaying information.

9. The line-set of claim 2 wherein the controller has a means for network communication.

10. The line-set of claim 9 wherein the network communication further comprises means for automated control and interrogation of the MEMS element.

11. A disposable line-set comprising:
a disposable length of tube; and
a disposable MEMS element connected to the tube.

12. The line-set of claim 11 further comprising a reusable controller operably connected to the MEMS element.

13. The line-set of claim 11 further comprising a power source operably connected to the MEMS element.

14. The line-set of claim 13 wherein the power source is disposable.

15. A medical line-set comprising:
a length of tube; and
a MEMS pump element attached to the tube.

16. A medical line-set comprising:
a length of tube having a first end adapted to be connected to a container and a second end adapted to be connected to another component, the tube having a MEMS element attached thereon.

17. The line-set of claim 16 further comprising a power source operably connected to the MEMS element.

18. The line-set of claim 16 further comprising a MEMS element controller operably connected to the MEMS element.

19. An infusion system comprising:
a length of tube having a first end adapted to be connected to a container and a second end adapted to be connected to a body, the tube having a MEMS element attached thereon.

20. The infusion system of claim 19 wherein the MEMS element is a flow sensor.

21. The infusion system of claim 19 wherein the MEMS element is a flow valve.

22. The infusion system of claim 19 wherein the MEMS element is a pressure sensor.

23. The infusion system of claim 19 wherein the MEMS element is a pump.

24. An infusion system comprising:
a length of tube having a first end adapted to be connected to a container and a second end adapted to be connected to a body, the tube having a MEMS element attached thereon, the MEMS element being controllable by a wireless controller.

25. The infusion system of claim 24 further comprising a power source operably connected to the MEMS element.

26. The infusion system of claim 24 wherein the controller has a means for network communication.

27. The infusion system of claim 24 wherein the tube and MEMS element are disposable.

28. The infusion system of claim 25 wherein the power source is disposable.

29. The infusion system of claim 24 wherein the MEMS element is remotely controllable by the wireless controller.

30. A medical line-set comprising:
a length of tube;
a MEMS pump element attached to the tube; and
a power source connected to the MEMS element.

31. The line-set of claim 30 wherein the power source is detachable from the MEMS element.

32. A medical line-set comprising:
a length of tube; and
a MEMS element adapted to be attached to the tube, the line-set capable of being implanted inside a body.

33. The medical line-set of claim 32 further comprising an implantable power source operably connected to the MEMS element.

34. The line-set of claim 32 further comprising a reusable, wireless MEMS element controller operably connected to the MEMS element.

35. A disposable medical infusion and draw line-set comprising:
a disposable tube;
a disposable electromechanical pump element connected to the tube;
a reusable pump controller operably connectable to the pump element; and
a disposable reservoir operably attached to the tube.

36. The system of claim 35 wherein the disposable reservoir has at least one valve.

37. The system of claim 36 wherein the valve is controlled remotely.

38. The system of claim 35 wherein the pump element is volumetric.

39. The system of claim 35 wherein the pump element is ambulatory.

40. The system of claim 35 wherein the pump element is wearable.

41. The system of claim 35 wherein the pump element is portable.

42. The system of claim 35 wherein the pump element includes a slide clamp.

43. A medical line-set comprising:
a tube having a first end adapted to be connected to a container and a second end adapted to be connected to another component;
a MEMS pump attached to the tube and configured to pump fluid from the container through the tube; and
a power source attached to the tubing and operably connected to the MEMS pump.

44. The medical line-set of claim 43 wherein the MEMS pump and the power source are contained within the tube.

45. A method for delivering a medication from a container to a patient, the method comprising the steps of:
providing tubing having a MEMS pump attached thereto, the MEMS pump being operably connected to a power supply, the tubing having a first end adapted to be in communication with the container and a second end adapted to be in communication with the patient; and
activating the power supply to power the pump wherein the medication is pumped by the MEMS pump from the container to the patient.

46. The method of claim 45 further comprising the step of controlling the MEMS pump with an external controller.

47. The method of claim 45 further comprising the step of discarding the tubing and MEMS pump after use.

48. A method of delivering fluid from a container comprising the steps of:
providing tubing having a MEMS pump attached thereto, the tubing having a first end adapted to be in communication with the container and a second end;
providing a controller having a power supply;
operably connecting the controller to the MEMS pump;
activating the controller to provide power to the MEMS pump; and
pumping fluid from the container and through the tubing.

49. The method of claim 48 wherein the controller controls the MEMS pump to deliver fluid at a predetermined rate.

50. The method of claim 48 wherein the controller is connected to the MEMS pump is operably connected to the controller by a wired connection.

51. The method of claim 48 wherein the controller is connected to the MEMS pump is operably connected to the controller by a wireless connection.

52. The method of claim 48 further comprising the step of providing a flow sensor that is attached to the tubing.

53. The method of claim 48 further comprising the step of providing a valve that is attached to the tubing.

54. The method of claim 48 further comprising the step of calibrating the MEMS pump with the controller.

55. The method of claim 48 further comprising the step of discarding the tube and MEMS pump after use.

56. A method of delivering a medication to a patient comprising the steps of:
providing an infusion system having a tubing having a MEMS pump connected thereto, the MEMS pump having a power supply, the tubing having a first end attached to a container containing a medication and a second end;
implanting the infusion system within the patient wherein the second end of the tube is positioned at a desired location;
providing a controller outside of the patient;
activating the controller to activate the MEMS pump; and
pumping fluid from the container and through the second end of the tube wherein the medication is adapted to be delivered to the desired location.

57. A system for infusion comprising:
a length of tube;
a MEMS element connected to the tube; and
means for controlling the MEMS element.

58. The system of claim 57 further comprising means for storing and displaying infusion data.

59. The system of claim 57 further comprising means for network communication.

60. The line set of claim 57 wherein the means for controlling the MEMS element is wireless.

61. The line set of claim 57 further comprising means for operably attaching a disposable power source to the MEMS element.

62. A medical line-set comprising:
a length of tube having a first and second end;
the tube having an attached MEMS element;
means for connecting the first end of the tube to a container; and
means for controlling fluid flow through the tube with the MEMS element.

63. The medical line-set of claim 62 wherein the MEMS element comprises a means for pumping.

64. The medical line-set of claim 62 further comprising means for sensing pressure.

65. The medical line-set of claim 62 further comprising means for sensing flow.

66. The medical line-set of claim 62 wherein the MEMS element comprises a flow valve.

67. The medical line-set of claim 62 wherein the MEMS element comprises means for supplying power.

68. The medical line-set of claim 62 further comprising means for implanting the line-set inside a body.

69. The medical line-set of claim 62 further comprising means for controlling the MEMS element with a wireless remote controller.

70. A medical line-set comprising:
a length of tube;
a MEMS element adapted to be attached to the tube, and
means for implanting the line-set inside a body.

71. A disposable medical infusion and draw line-set comprising:
a disposable tube;
a disposable micro-electromechanical pump element connected to the tube;
means for operably connecting a reusable pump controller to the pump element; and
means for operably attaching a disposable reservoir to the tube.

72. The system of claim 71 wherein the disposable reservoir has at least one valve.

73. The system of claim 72 wherein the valve is controlled remotely.
